(19) United States
(12) Patent Application Publication
(10) Pub. No.: US 2015/0238216 A1
(43) Pub. Date: Aug. 27, 2015

(54) FLUID EJECTING APPARATUS AND MEDICAL DEVICE

(71) Applicant: SEIKO EPSON CORPORATION, Tokyo (JP)

(72) Inventors: Kazuaki UCHIDA, Fujimi-machi (JP); Junichi KARASAWA, Shimosuwa-machi (JP); Takahiro MATSUZAKI, Shiojiri-shi (JP)

(21) Appl. No.: 14/629,103

(22) Filed: Feb. 23, 2015

(30) Foreign Application Priority Data
Feb. 24, 2014 (JP) 2014-032421

(51) Int. Cl. A61B 17/3203

(52) U.S. Cl. CPC A61B 17/3203; A61B 2017/32032; A61B 2017/32035

(57) ABSTRACT

A fluid ejecting apparatus which ejects a fluid, includes: an ejection pipe which has an opening through which the fluid is ejected; a fluid chamber which is communicated with the ejection pipe and accommodates the fluid therein; a bubble generating unit which generates a bubble in the fluid within the fluid chamber; a supply flow path which is communicated with the fluid chamber; an opening and closing unit which is provided in the supply flow path and opens and closes the supply flow path; a fluid supply portion which supplies the fluid to the fluid chamber through the supply flow path by pressurizing the fluid; and a driving control portion which controls driving of the bubble generating unit. The driving control portion performs control such that a bubble is generated by the bubble generating unit after the supply flow path is opened by the opening and closing unit.

This application claims the benefit of Japanese Patent Application No. 2014-032421, filed on Feb. 24, 2014. The content of the aforementioned application is incorporated herein by reference in its entirety.

### BACKGROUND

1. Technical Field

The present invention relates to a fluid ejecting apparatus and a medical device.

2. Related Art

A medical device disclosed in JP-A-2008-82202 is known as a medical device for treating a lesion area by applying a fluid, which is ejected, to the lesion area, for example. In the fluid ejecting apparatus disclosed in JP-A-2008-82202, the capacity of a fluid chamber is increased and decreased by driving a capacity changing unit, and a pulsating flow (pulse flow) is ejected from an ejection pipe.

A fluid ejecting apparatus is used as, for example, a scalpel for medical use, and therefore, is required to have a stable strength (force) of a pulsating flow. Particularly, in order to improve a comfort of an operator during use, it has been requested that the fluid ejecting apparatus eject a pulsating flow with an adequate force immediately after the start of the ejection.

In addition, in the fluid ejecting apparatus in the related art, miniaturization, low cost, resource saving, easy manufacturing, improvement in usability, and the like have been requested.

### SUMMARY

An advantage of some aspects of the invention is to solve at least a part of the problems described above, and the invention can be implemented as the following aspects.

(1) An aspect of the invention provides a fluid ejecting apparatus which ejects a fluid. The fluid ejecting apparatus includes an ejection pipe which has an opening through which the fluid is ejected; a fluid chamber which is communicated with the ejection pipe and accommodates the fluid therein; a bubble generating unit which generates a bubble in the fluid within the fluid chamber; a supply flow path which is communicated with the fluid chamber; an opening and closing unit which is provided in the supply flow path and opens and closes the supply flow path; a fluid supply portion which supplies the fluid to the fluid chamber through the supply flow path by pressurizing the fluid; and a driving control portion which controls driving of the bubble generating unit. The driving control portion performs control such that a bubble is generated by the bubble generating unit after the supply flow path is opened by the opening and closing unit. Immediately after the supply flow path is opened by the opening and closing unit, the pressure of the fluid supplied from the supply flow path temporarily increases and the pressure in the fluid chamber also temporarily increases. According to the aspect of the fluid ejecting apparatus, a bubble is not generated by the bubble generating unit when the supply flow path is opened by the opening and closing unit. Accordingly, it is possible to suppress the generation of a bubble using the bubble generating unit in a state in which the pressure of the fluid within the fluid chamber is temporarily increased. As a result, it is possible to eject the pulsating flow with an adequate force immediately after the start of the ejection.

(2) In the fluid ejecting apparatus according to the aspect described above, the driving control portion may perform control such that a driving voltage is applied to the bubble generating unit after the supply flow path is opened by the opening and closing unit. According to this aspect of the fluid ejecting apparatus, the bubble generating unit starts driving due to the driving voltage which is applied to the bubble generating unit after a start of supply of fluid to the fluid chamber. Therefore, it is possible to suppress the bubble generating unit from being driven in a state of insufficient fluid in the fluid chamber.

(3) In the fluid ejecting apparatus according to the aspect described above, the driving control portion may perform control such that a driving voltage is applied to the bubble generating unit when a predetermined amount of time elapses after the supply flow path is opened by the opening and closing unit. When a predetermined amount of time elapses after the supply flow path is opened, the temporarily increased pressure in the fluid chamber decreases and is stabilized at an almost constant value. According to this aspect of the fluid ejecting apparatus, the bubble generating unit starts driving due to the driving voltage which is applied to the bubble generating unit when a predetermined amount of time elapses after the supply flow path is opened. Therefore, it is possible to eject a pulsating flow with an adequate force immediately after the start of the ejection.

(4) In the fluid ejecting apparatus according to the aspect described above, the supply flow path may include an elastic pipe line, and the opening and closing unit may include a pinch valve which closes the supply flow path by pressing the elastic pipe line from the outside. According to this aspect of the fluid ejecting apparatus, it is possible to open and close the supply flow path without bringing the opening and closing unit into contact with a fluid within the pipe line, and therefore, it is possible to improve sanitation of the fluid.

(5) Another aspect of the invention provides a medical device using the fluid ejecting apparatus described above. According to this aspect of the invention, it is possible to provide a highly reliable medical device.

The plurality of constituents provided in each aspect of the invention described above are not essential. Moreover, in order to solve a part or all of the problems described above, or to achieve a part or all of the effects described in the present specification it is possible to appropriately perform modification, deletion, replacement with other new constituents with respect to a part of constituents of the plurality of constituents, or to perform deletion of a part of limited contents. In addition, in order to solve a part or all of the problems described above, or to achieve a part or all of the effects described in the present specification it is also possible to combine a part or all of the technical features included in an aspect of the invention described above with a part or all of the technical features included in another aspect of the invention described above to make an independent aspect of the invention.

For example, an aspect of the invention can be implemented as an apparatus provided with one or more elements among the seven elements including the ejection pipe, the fluid chamber, the bubble generating unit, the supply flow path, the opening and closing unit, the fluid supply portion, and the driving control portion. That is, the apparatus may or may not have the ejection pipe. In addition, the apparatus may or may not have the fluid chamber. In addition, the apparatus may or may not have the bubble generating unit. In addition, the apparatus may or may not have the supply flow path. In addition, the apparatus may or may not have the opening and closing unit. In addition, the apparatus may or may not have the fluid supply portion. In addition, the apparatus may or may not have the driving control portion. The ejection pipe may be configured as, for example, an ejection pipe having an opening which ejects the fluid. The fluid chamber may be configured as, for example, a fluid chamber which is communicated with the ejection pipe and accommodates the fluid therein. The bubble generating unit may be configured as, for example, a bubble generating unit which generates a bubble in the fluid within the fluid chamber. The supply flow path may be configured as, for example, a supply flow path which is communicated with the fluid chamber. The opening and closing unit may be configured as, for example, an opening and closing unit which is provided in the supply flow path and opens and closes the supply flow path. The fluid supply portion may be configured as, for example, a fluid supply portion which supplies the fluid to the fluid chamber through the supply flow path by pressurizing the fluid. The driving control portion may be configured as, for example, a driving control portion which performs control such that a bubble is generated by the bubble generating unit after the supply flow path is opened by the opening and closing unit. Such an apparatus can be implemented as, for example, a fluid ejecting apparatus which ejects a fluid, but can also be implemented as apparatuses other than the fluid ejecting apparatus which ejects a fluid. According to the aspect, it is possible to achieve at least one of various subjects including miniaturization of the apparatus, low cost, resource saving, easy manufacturing, and improvement of usability. A part or all of the technical features of all of the aspects of the above-described fluid ejecting apparatus which ejects a fluid can be applied to this apparatus.

The invention can be implemented in various forms other than the apparatus. For example, it is possible to implement the invention in forms such as a method of ejecting a fluid or a method of manufacturing the fluid ejecting apparatus.

### BRIEF DESCRIPTION OF THE DRAWINGS

The invention will be described with reference to the accompanying drawings, wherein like numbers reference like elements.

FIG. 1 is an explanatory view showing a configuration of a fluid ejecting apparatus according to an embodiment of the invention.

FIG. 2 is a cross-sectional view showing an internal structure of a handpiece of which a portion is enlarged.

FIG. 3 is an explanatory view showing a measurement result of the pressure of a fluid within a fluid chamber immediately after opening a valve.

FIG. 4 is an explanatory view showing change in a driving voltage applied to an electromagnetic wave beam source.

FIG. 5 is an explanatory view showing an example of a timing chart when a foot switch is turned on.

FIG. 6 is a flowchart showing a process when the foot switch is turned on.

### DESCRIPTION OF EXEMPLARY EMBODIMENTS

Next, embodiments of the invention will be described in order of an embodiment and modification examples.

### A. Embodiment

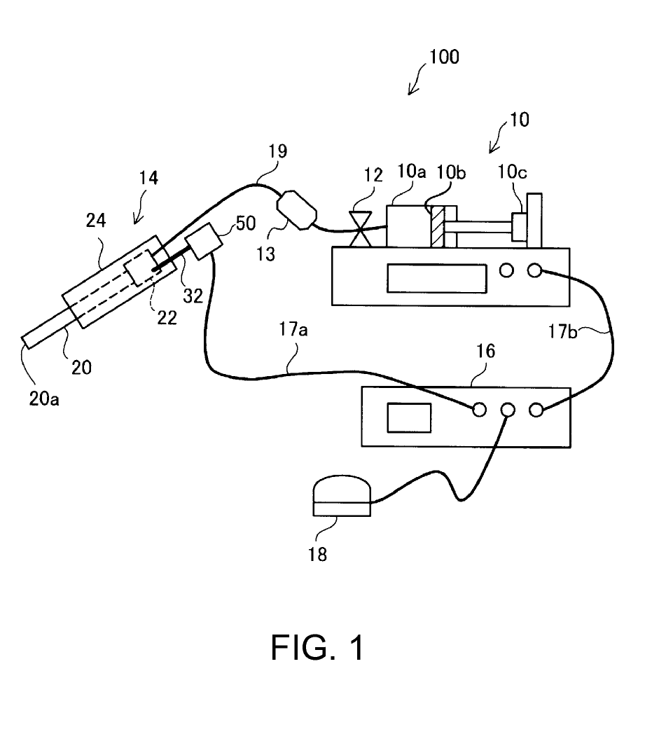

FIG. 1 is an explanatory view showing a configuration of a fluid ejecting apparatus 100 according to an embodiment of the invention. The fluid ejecting apparatus 100 of the present embodiment is a medical device utilized in medical institutions and has a function of performing incision or excision of a lesion area as a scalpel by ejecting a fluid to the lesion area.

The fluid ejecting apparatus 100 includes a fluid supply portion 10, a handpiece 14, a control portion 16, and afoot switch 18. The fluid supply portion 10 and the handpiece 14 are connected through a connection tube 19 made of resin.

The connection tube 19 is provided with a valve 12 as an opening and closing unit which opens and closes a flow path, and a filter 13 which removes foreign bodies, bacteria, bubbles, or the like within the connection tube 19.

The fluid supply portion 10 supplies a fluid to the handpiece 14 through the connection tube 19. In the present embodiment, the fluid supply portion 10 is a syringe type pump and is provided with a cylindrical syringe 10a, a piston 10b which changes the capacity of the syringe 10a, and an actuator 10c which moves the piston 10b within the syringe 10a.

The syringe 10a accommodates physiological saline as a fluid supplied to the handpiece 14. However, the syringe 10a may accommodate other fluids, for example, pure water or a liquid medicine, which are not harmful even when ejected to a lesion area, instead of the physiological saline.

The piston 10b is movable within the syringe 10a by being operated by the actuator 10c and can change the capacity of the syringe 10a. In the present embodiment, the piston 10b is formed of resin in order to enhance airtightness of the syringe 10a.

The valve 12 is an opening and closing unit which opens and closes the flow path, and a pinch valve which closes the flow path within the connection tube 19 by pinching the elastic connection tube 19 from the outside is used in the present embodiment. Accordingly, in the present embodiment, it is possible to open and close the flow path without bringing the valve into contact with the fluid within the connection tube 19, and to sanitarily maintain the fluid within the flow path. In addition, it is possible to reuse the pinch valve even if the used or old connection tube 19 is discarded to be replaced with a new connection tube 19. However, other types of the valves such as a gate valve or a ball valve may be used as the valve 12.

In the present embodiment, a sensor which measures the pressure of the fluid within the syringe 10a is provided in the fluid supply portion 10. The actuator 10c is controlled such that the pressure of the fluid within the syringe 10a becomes a predetermined pressure when the valve 12 is closed. When the fluid supply portion 10 receives a command to supply a fluid to the handpiece 14 from the control portion 16, the fluid supply portion 10 opens the valve 12 and moves the piston 10b at a predetermined speed by operating the actuator 10c. As a result, the capacity of the syringe 10a is reduced and the fluid within the syringe 10a is pushed out to the connection tube 19.

The handpiece 14 is an instrument, which is operated by an operator by being held by hand, and has a fluid ejection pipe 20, a pulsation imparting portion 22, and a housing 24. When the fluid is supplied from the fluid supply portion 10, pulsation is imparted to the supplied fluid by the pulsation imparting portion 22, and the handpiece 14 ejects the fluid (pulsating flow), to which the pulsation is imparted, at a high speed from an opening 20a at a tip end of the fluid ejection pipe 20. An operator performs incision or excision of a lesion area by applying the fluid ejected from the handpiece 14 to the lesion area of a patient.

The control portion 16 controls the flow rate of the fluid supplied to the handpiece 14 by applying a driving voltage to the pulsation imparting portion 22 through a voltage applying cable 17a and controlling the fluid supply portion 10 and the valve 12 through a control cable 17b.

The foot switch 18 is a switch, which is operated by the foot of an operator, and is connected to the control portion 16. When the operator turns on the foot switch 18, a driving voltage is applied to the pulsation imparting portion 22 and the valve 12 is opened, and thereby the fluid supply portion 10 starts to supply the fluid. As a result, the fluid (pulsating flow) to which the pulsation is imparted is ejected at a high speed from the opening 20a of the tip end of the fluid ejection pipe 20 of the handpiece 14.

The fluid ejecting apparatus 100 of the present embodiment controls the supply of fluid to the handpiece 14 by opening and closing the valve 12, and therefore, is excellent in responsiveness with respect to an operation of an operator.

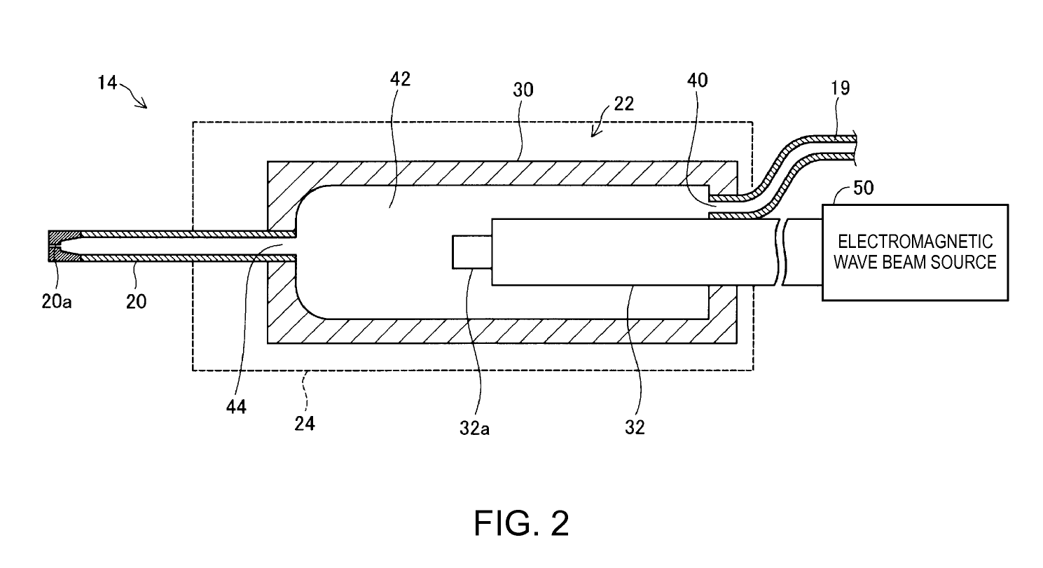

FIG. 2 is a cross-sectional view showing an internal structure of the handpiece 14 of which a portion is enlarged. The pulsation imparting portion 22 which imparts pulsation to the fluid supplied from the fluid supply portion 10 is provided inside the housing 24 of the handpiece 14. The pulsation imparting portion 22 is provided with a pipe 30 and an optical fiber 32 which is disposed within the pipe 30.

An inlet flow path 40, a fluid chamber 42, and an outlet flow path 44 are formed inside the pipe 30 as flow paths through which the fluid supplied from the fluid supply portion 10 passes. The inlet flow path 40 is formed in a rear end portion of the pipe 30 and the outlet flow path 44 is formed in a tip end portion of the pipe 30. The fluid chamber 42 is formed in an inner portion of the pipe 30. The connection tube 19 is connected to the inlet flow path 40 and the fluid ejection pipe 20 is connected to the outlet flow path 44.

The optical fiber 32 extends to the outside from the rear end portion of the pipe 30 and is connected to an electromagnetic wave beam source 50. That is, the electromagnetic wave beam source 50 is provided outside the handpiece 14 and is communicated with the inside of the fluid chamber 42 through the optical fiber 32. The electromagnetic wave beam source 50 outputs an electromagnetic wave beam when a driving voltage is applied from the control portion 16. A coherent optical maser with high directivity and convergence is output as the electromagnetic wave beam. The wavelength of the electromagnetic wave beam is 2.1 μm in the present embodiment and the electromagnetic wave beam is an optical maser in an infrared region. The output electromagnetic wave beam is introduced into the fluid chamber 42 inside the pipe 30 by the optical fiber 32.

The fluid chamber 42 is filled with the fluid supplied from the fluid supply portion 10, and the electromagnetic wave beam which is introduced by the optical fiber 32 is emitted to the fluid. When the electromagnetic wave beam is emitted to the fluid, energy of the electromagnetic wave beam is absorbed in the fluid which is then vaporized. In the present embodiment, the output of the electromagnetic wave beam is intermittently performed, and therefore, the vaporization also intermittently occurs. Accordingly, a vapor bubble is generated around a tip end 32a of the optical fiber 32. The internal pressure of the fluid chamber 42 rapidly increases due to the generation of the vapor bubble, and the fluid which has been pressed by the pressure passes through the outlet flow path 44 and is ejected from a nozzle 20a (opening 20a) at the tip end of the fluid ejection pipe 20, as a pulse jet at once. At this time, the ejection speed of the pulse jet ejected from the nozzle 20a is high and the pulse jet is capable of excising a tissue. The electromagnetic wave beam source 50 and the optical fiber 32 correspond to the “bubble generating unit” disclosed in the section of “Summary”.

The driving voltage applied to the electromagnetic wave beam source 50 from the control portion 16 is a pulse wave which is repeatedly turned on (maximum voltage) and turned off (0 V) at a predetermined frequency (for example, 10 Hz). Accordingly, the output of the electromagnetic wave beam due to the electromagnetic wave beam source 50 is intermittently performed. The OFF voltage is denoted as 0 V. However, the OFF voltage may not be 0 V as long as the OFF voltage is a voltage smaller than the maximum voltage in an ON state.

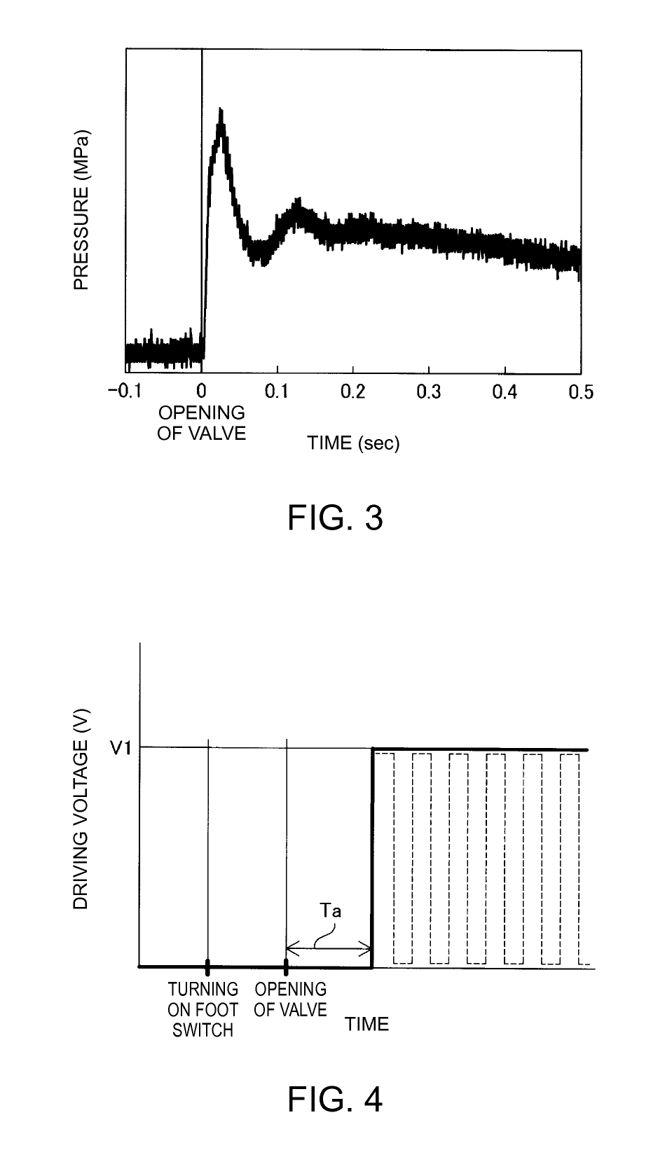

FIG. 3 is an explanatory view showing a measurement result of the pressure of a fluid within the fluid chamber 42 immediately after opening the valve 12. In FIG. 3, the horizontal axis indicates the time and the longitudinal axis indicates the pressure of the fluid within the fluid chamber 42. In addition, when measuring the pressure shown in FIG. 3, the electromagnetic wave beam source 50 is not driven.

As shown in FIG. 3, it was confirmed that when the valve 12 is opened at a time 0 and the fluid supply portion 10 starts the supply of the fluid, the pressure of the fluid within the fluid chamber 42 decreases after showing a temporarily high value immediately after the opening of the valve 12, and then, is stabilized at an almost constant value.

One of the reasons can be considered as follows. When the valve 12 is opened in a state in which a high pressure is loaded on the syringe 10a, the fluid is made to flow to the handpiece 14 at once. However, there is a factor causing flow path resistance such as the filter 13 in the middle of the flow path from the fluid supply portion 10 to the fluid chamber 42 of the handpiece 14, and therefore, the fluid is temporarily blocked. In contrast, it is considered that it is because the supply of the fluid from the syringe 10a is continued, and thus, the pressure of the side of the resistance factor such as the filter 13 temporarily increases, and the increased pressure flows into the handpiece 14. In addition, it is also considered that the opening of the valve 12 is regarded as performing step input in the transmission process of the pressure, and therefore, the high pressure is generated within the fluid chamber 42 of the handpiece 14 immediately after the opening of the valve 12.

FIG. 4 is an explanatory view showing change in the driving voltage applied to the electromagnetic wave beam source 50. The dashed line in the drawing is an example of the driving voltage. The driving voltage applied to the electromagnetic wave beam source 50 is repeatedly turned on (maximum voltage) and turned off (0 V) at a predetermined frequency (for example, 10 Hz). The driving voltage is drawn at a lower frequency than the actual frequency in order to facilitate the understanding of transition of the maximum voltage. The solid line in FIG. 4 shows the transition of the maximum voltage of the driving voltage. In addition, the scale of the horizontal axis in FIG. 4 is different from that in FIG. 3. Hereinafter, only the transition of the maximum voltage of the driving voltage is shown in the drawing which shows the change in the driving voltage applied to the electromagnetic wave beam source 50.

As shown in FIG. 4, when the foot switch 18 is turned on, the control portion 16 opens the valve 12 and operates the actuator 10c of the fluid supply portion 10 to start a supply of fluid. Furthermore, the control portion 16 applies the driving voltage to the electromagnetic wave beam source 50 when a predetermined amount of time Ta elapses after the opening of the valve 12. The driving voltage is controlled so as to instantly reach a predetermined voltage V1, which is a maximum voltage, immediately after the start of the applying of the driving voltage. Accordingly, as shown in FIG. 4, the driving voltage does not reach the predetermined voltage V1 at the time of opening of the valve 12.

Specifically, in the present embodiment, the driving voltage is not applied to the electromagnetic wave beam source 50 immediately after the opening of the valve 12 while the pressure of the fluid within the fluid chamber 42 is temporarily increased. The predetermined voltage V1 is applied to the electromagnetic wave beam source 50 as the driving voltage after the lapse of a predetermined amount of time Ta (for example, 0.1 seconds) while the pressure of the fluid within the fluid chamber 42 is stabilized at an almost constant value. Accordingly, it is possible to suppress the ejection of a pulsating flow with a strong force immediately after the start of the ejection. That is, according to the present embodiment, it is possible to eject a pulsating flow with an adequate force immediately after the start of the ejection.

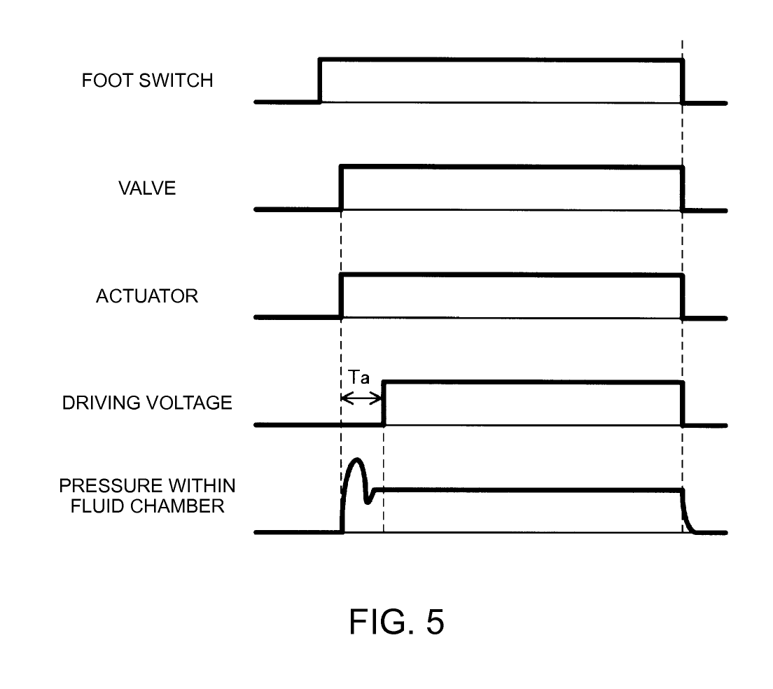

FIG. 5 is an explanatory view showing an example of a timing chart when the foot switch 18 is turned on. The control portion 16 starts to apply a driving voltage with the turning on of the foot switch 18 as a trigger. Furthermore, the control portion 16 opens the valve 12 and operates the actuator 10c with the turning on of the foot switch 18 as a trigger. Then, the pressure of the fluid within the fluid chamber 42 temporarily increases immediately after the opening of the valve 12, and then is stabilized at an almost constant value. As described above, in the present embodiment, the driving voltage is not applied to the electromagnetic wave beam source 50 during the period when the pressure of the fluid within the fluid chamber 42 is temporarily increased, and a predetermined voltage V1 is applied thereto as the driving voltage after the pressure of the fluid within the fluid chamber 42 is stabilized at an almost constant value.

In contrast, the control portion 16 closes the valve 12 and stops the actuator 10c with turning off of the foot switch 18 as a trigger to stop the applying of the driving voltage to the electromagnetic wave beam source.

A comfort of an operator during use is improved when the time from the turning on of the foot switch 18 until the driving voltage reaches a predetermined voltage V1 which is the maximum voltage is short. Accordingly, it is preferable that the time from the turning on of the foot switch 18 until the maximum voltage of the driving voltage reaches the predetermined voltage V1 be shorter than or equal to 0.2 seconds.

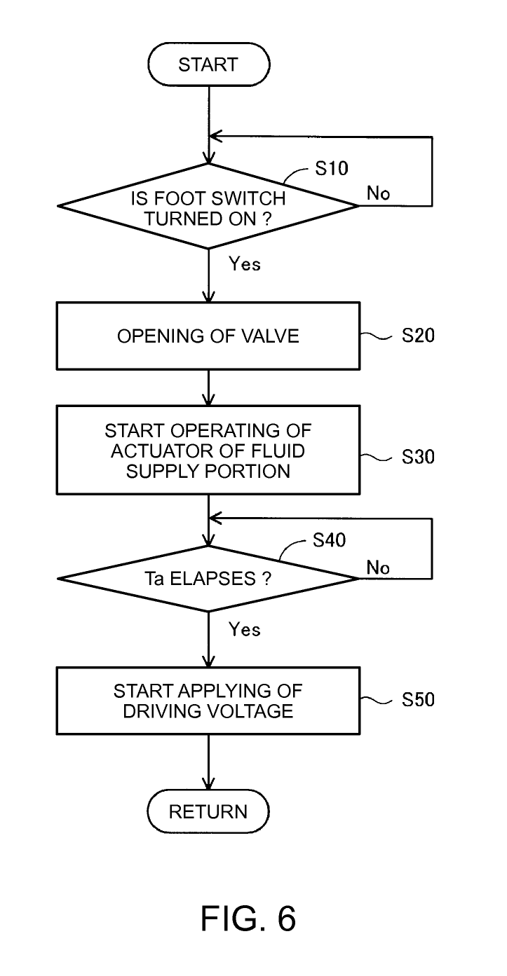

FIG. 6 is a flowchart showing a process when the foot switch 18 is turned on. The control portion 16 determines whether the foot switch 18 is turned on (step S10). When the foot switch 18 is turned on, the control portion 16 opens the valve 12 (step S20), and then, operates the actuator 10c of the fluid supply portion 10 (step S30). The control portion 16 determines whether a predetermined amount of time Ta elapses from the opening of the valve 12 (step S40). When a predetermined amount of time Ta elapses, the control portion starts to apply a driving voltage to the electromagnetic wave beam source 50 (step S50).

In this manner, according to the present embodiment, the predetermined voltage V1 is not applied to the electromagnetic wave beam source 50 as the driving voltage immediately after the opening of the valve 12 while the pressure of the fluid within the fluid chamber 42 is temporarily increased, and therefore, it is possible to eject the pulsating flow with an adequate force immediately after the start of the ejection.

As shown in FIGS. 3 and 5, after the lapse of a predetermined amount of time Ta from the opening of the valve 12, the temporarily increased pressure within the fluid chamber 42 decreases and is stabilized at an almost constant value. In the present embodiment, after the lapse of a predetermined amount of time Ta from the opening of the valve 12, that is, after the temporarily increased pressure of the fluid within the fluid chamber 42 decreases and is stabilized at an almost constant value, the electromagnetic wave beam source 50 starts driving, and therefore, it is possible to eject a pulsating flow with an adequate force immediately after the start of the ejection.

Furthermore, in the present embodiment, the electromagnetic wave beam source 50 starts driving after the valve 12 is opened and the fluid starts to be supplied to the fluid chamber 42. Therefore, it is possible to suppress the driving of the electromagnetic wave beam source 50 in a state of insufficient fluid in the fluid chamber 42.

According to FIG. 3, in the fluid ejecting apparatus 100 of the present embodiment, it can be understood that the pressure of the fluid within the fluid chamber 42 is stabilized at an almost constant value about 0.1 seconds after the opening of the valve 12. Accordingly, it is preferable that the control portion 16 of the present embodiment perform control such that a driving voltage is applied to the electromagnetic wave beam source 50 about 0.1 seconds after the opening of the valve 12 as described above. However, the time required for stabilization of the pressure of the fluid within the fluid chamber 42 at an almost constant value varies depending on the configuration of the fluid ejecting apparatus 100. Accordingly, it is preferable that the time required to start applying of the driving voltage from the opening of the valve 12 be appropriately set depending on the configuration of the fluid ejecting apparatus 100.

### B. Modification Examples

The invention is not limited to the above-described embodiment and can be implemented in various forms within the scope not departing from the gist thereof. For example, the following modification can be made.

### Modification Example 1

In the above-described embodiment, the fluid ejecting apparatus 100 is used as a medical device. In contrast, in the modification example, the fluid ejecting apparatus 100 may be used as devices other than the medical device. For example, the fluid ejecting apparatus 100 may be used as a cleaning device for removing dirt of an object by applying an ejected fluid to the object, or a depiction device for drawing characters or pictures using an ejected fluid.

### Modification Example 2

In the above-described embodiment, liquid is used as the fluid ejected from the fluid ejecting apparatus 100. In contrast, in the modification example, a gas may be used as the fluid ejected from the fluid ejecting apparatus 100.

### Modification Example 3

In the above-described embodiment, a bubble is generated in the fluid chamber 42 by a coherent optical maser in an infrared region which has a wavelength of 2.16 μm, as the bubble generating unit. In contrast, the bubble generating unit may be configured such that a bubble is generated in the fluid chamber 42 through optical masers with other wavelengths or electromagnetic wave beams other than the optical maser. For example, an optical maser in a visible region or an optical maser in an ultraviolet region may be used instead of the optical maser in the infrared region. A coherent microwave may be used as the electromagnetic wave beam other than the optical maser, for example. In this case, a waveguide is employed instead of the optical fiber. In addition, the bubble generating unit may generate a bubble in the fluid chamber 42 using a microwave or a far-infrared ray which is not coherent. Furthermore, the bubble generating unit may generate a bubble in the fluid chamber 42 using units other than those emitting the electromagnetic wave beam. Other units may be used to generate a bubble in the fluid chamber 42 through instantaneous heating using an electric heating element such as a resistance heater or a ceramic heater, or to generate a bubble using discharge from an electrode.

### Modification Example 4

In the above-described embodiment or modification examples, the energy source for generating a bubble is provided outside the handpiece 14. However, the energy source for generating a bubble may be configured to be provided inside the handpiece. For example, it is possible to have a configuration in which the electric heating element is provided inside the handpiece.

### Modification Example 5

In the above-described embodiment or modification examples, the timing for opening the valve 12 or starting the operation of the actuator 10c may be immediately after the foot switch 18 is turned on. Accordingly, it is possible to shorten the time until the maximum voltage of a driving voltage reaches a predetermined voltage V1.

### Modification Example 6

In the above-described embodiment or modification examples, a configuration may be employed in which a driving voltage is applied to the electromagnetic wave beam source 50 after the lapse of a predetermined amount of time Ta from the opening of the valve 12 and the driving voltage immediately after the starting of the application reaches a predetermined voltage V1 as the maximum voltage. However, instead of the configuration, a configuration may be employed in which the driving voltage is applied to the electromagnetic wave beam source 50 after the lapse of a predetermined amount of time Ta from the opening of the valve 12 and the driving voltage immediately after the starting of the application reaches a predetermined voltage V1 by gradually becoming a greater value. Furthermore, a configuration may also be employed in which the driving voltage is applied to the electromagnetic wave beam source 50 before the opening of the valve 12 (for example, at the time point when the foot switch 18 is turned on) and the driving voltage immediately after the starting of the application reaches a predetermined voltage V1 by gradually becoming a greater value, and the time point at which the driving voltage reaches a predetermined voltage V1 comes later than the time point at which the valve 12 is opened. In short, it is possible to have any configuration as long as the driving voltage reaches a predetermined voltage V1 after the opening of the valve 12. According to the configurations, similarly to the above-described embodiment, it is possible to suppress the ejection of the pulsating flow with a strong force immediately after the start of the ejection.

### Modification Example 7

In the above-described embodiment or modification examples, a configuration may be employed such that the driving of the bubble generating unit is controlled by applying a driving voltage to the electromagnetic wave beam source 50. However, it is unnecessary for the invention to always be limited to the control of the voltage. For example, a configuration may be employed such that the output of the constant electromagnetic wave beam due to the electromagnetic wave beam source 50 is performed, and the emission of the electromagnetic wave beam to the inside of the fluid chamber 42 is controlled by proving an optical shutter between the electromagnetic wave beam source 50 and the optical fiber 32 and by driving the optical shutter.

### Modification Example 8

In the above-described embodiment, a switch which is operated by hand instead of by the foot switch 18 which is operated by the foot may be provided. The switch which is operated by the hand may be provided in, for example, the handpiece 14.

### Modification Example 9

In the above-described embodiment, a part of functions implemented by the software may be implemented by the hardware, or a part of functions which is implemented by the hardware may be implemented by the software.

The invention is not limited to the above-described embodiment, examples, or modification examples and can be implemented in various configurations within the scope not departing from the gist thereof. For example, it is possible to appropriately replace the technical features in the embodiment, the examples, or the modification examples corresponding to the technical features in each of the forms disclosed in the section of Summary with others or to appropriately combine them together in order to solve a part or all of the problems described above, or to achieve a part or all of the effects described above. In addition, it is possible to appropriately delete the technical features which are not described in the present specification as essential features.

What is claimed is:

1. A fluid ejecting apparatus which ejects a fluid, comprising:
an ejection pipe which has an opening through which the fluid is ejected;
a fluid chamber which is communicated with the ejection pipe and accommodates the fluid therein;
a bubble generating unit which generates a bubble in the fluid within the fluid chamber;
a supply flow path which is communicated with the fluid chamber;
an opening and closing unit which is provided in the supply flow path and opens and closes the supply flow path;
a fluid supply portion which supplies the fluid to the fluid chamber through the supply flow path by pressurizing the fluid; and
a driving control portion which controls driving of the bubble generating unit,
wherein the driving control portion performs control such that a bubble is generated by the bubble generating unit after the supply flow path is opened by the opening and closing unit.

2. The fluid ejecting apparatus according to claim 1,
wherein the driving control portion performs control such that a driving voltage is applied to the bubble generating unit after the supply flow path is opened by the opening and closing unit.

3. The fluid ejecting apparatus according to claim 1,
wherein the driving control portion performs control such that a driving voltage is applied to the bubble generating unit when a predetermined amount of time elapses after the supply flow path is opened by the opening and closing unit.

4. The fluid ejecting apparatus according to claim 1,
wherein the supply flow path includes an elastic pipe line, and
wherein the opening and closing unit includes a pinch valve which closes the supply flow path by pressing the elastic pipe line from the outside.

5. A medical device using the fluid ejecting apparatus according to claim 1.

6. A medical device using the fluid ejecting apparatus according to claim 2.

7. A medical device using the fluid ejecting apparatus according to claim 3.

8. A medical device using the fluid ejecting apparatus according to claim 4.
